(19) United States
(12) Patent Application Publication
(10) Pub. No.: US 2025/0135201 A1
(43) Pub. Date: May 1, 2025

(54) SELECTIVE NEUROSTIMULATION OF PERIPHERAL NERVES WITH REDUCED OFF-TARGET ACTIVATION

(71) Applicant: BOARD OF TRUSTEES OF THE UNIVERSITY OF ARKANSAS, Little Rock, AR (US)

(72) Inventors: Ranu Jung, Fayetteville, AR (US); James Abbas, Fayetteville, AR (US); Arianna Ortega Sanabria, Fayetteville, AR (US); Anil Thota, Bentonville, AR (US)

(73) Assignee: BOARD OF TRUSTEES OF THE UNIVERSITY OF ARKANSAS, Little Rock, AR (US)

(21) Appl. No.: 18/927,691

(22) Filed: Oct. 25, 2024

Related U.S. Application Data
(60) Provisional application No. 63/545,717, filed on Oct. 25, 2023.

(51) Int. Cl. A61N 1/36; A61N 1/05; A61N 1/372

(52) U.S. Cl. CPC A61N 1/36053; A61N 1/0556; A61N 1/36139; A61N 1/36146; A61N 1/37247

(57) ABSTRACT

Embodiments pertain to a neurostimulation system operable for providing fascicular nerve fiber stimulation to one or more nerves of a subject. The neurostimulation systems may include: (1) one or more electrodes operational to associate with at least one fascicular nerve fiber region of a subject; (2) a stimulator in electrical communication with the electrodes and operational to provide one or more electrical pulses to the electrodes to stimulate at least one fascicular nerve fiber region; and (3) a signal processing unit in electrical communication with the electrodes and the stimulator, and operational to measure on-target and off-target activation by the stimulated electrodes. Further embodiments pertain to methods of providing at least one fascicular nerve fiber stimulation to one or more nerves of a subject by utilizing the disclosed neurostimulation systems. The stimulation may achieve intrafascicular selectivity.

### CROSS-REFERENCE TO RELATED APPLICATIONS

This application claims priority to U.S. Provisional Patent Application No. 63/545,717, filed on Oct. 25, 2023. The entirety of the aforementioned application is incorporated herein by reference.

### STATEMENT REGARDING FEDERALLY SPONSORED RESEARCH

This invention was made with government support under R01EB027584, awarded by the National Institutes of Health. The government has certain rights in the invention.

### BACKGROUND

A need exists for more selective systems and methods for stimulating nerves. Numerous embodiments of the present disclosure aim to address the aforementioned need.

### SUMMARY

Embodiments of the present disclosure pertain to a neurostimulation system operable for providing at least one fascicular nerve fiber stimulation to one or more nerves of a subject. The neurostimulation systems of the present disclosure may include: (1) one or more electrodes operational to associate with at least one fascicular nerve fiber region of a subject; (2) a stimulator in electrical communication with the electrodes and operational to provide one or more electrical pulses to the electrodes to stimulate at least one fascicular nerve fiber region; and (3) a signal processing unit in electrical communication with the electrodes and the stimulator, and operational to measure on-target and off-target activation by the stimulated electrodes.

In some embodiments, the stimulator also includes a pulse processing unit in electrical communication with the signal processing unit and operational to adjust one or more electrode stimulation parameters based on measurements from the signal processing unit. In some embodiments, the stimulator also includes a pulse generator in electrical communication with the pulse processing unit and the electrodes and operational to provide one or more adjusted electrical pulses to electrodes based on one or more adjusted electrode stimulation parameters from the pulse processing unit. In some embodiments, the pulse processing unit also includes a controller operational to control one or more stimulation parameters by the electrodes.

In some embodiments, the signal processing unit also includes one or more sensors operational to assess physiological and neural responses from a subject due to the neurostimulation. In some embodiments, the signal processing unit is operational to measure bio-signals from the sensors.

Additional embodiments of the present disclosure pertain to methods of providing at least one fascicular nerve fiber stimulation to one or more nerves of a subject. In some embodiments, the methods of the present disclosure may utilize the neurostimulation systems of the present disclosure to provide the nerve fiber stimulation. In some embodiments, the methods of the present disclosure include: (1) associating at least one fascicular nerve fiber region of the nerve with one or more electrodes; (2) utilizing a stimulator in electrical communication with the electrodes to provide one or more electrical pulses to the electrodes and thereby stimulate the fascicular nerve fiber region; and (3) utilizing a signal processing unit in electrical communication with the electrodes and the stimulator to measure on-target and off-target activation by the stimulated electrodes. In some embodiments, the methods of the present disclosure also include a step of (4) utilizing a pulse processing unit in electrical communication with the signal processing unit to adjust one or more electrode stimulation parameters based on measurements from the signal processing unit. In some embodiments, the methods of the present disclosure also include a step of (5) utilizing a pulse generator in electrical communication with the pulse processing unit to provide the adjusted electrical pulses to the electrodes based on one or more adjusted electrode stimulation parameters from the pulse processing unit.

### DESCRIPTION OF THE DRAWINGS

A better understanding of the present invention can be obtained when the following detailed description is considered in conjunction with the following drawings, in which:

FIG. 1A illustrates an architecture of a neurostimulation system in accordance with various embodiments of the present disclosure.

FIG. 1B illustrates a method of providing fascicular nerve fiber stimulation in accordance with various embodiments of the present disclosure.

FIG. 1C shows nerve diagram representations for intraneural and intrafascicular selectivity. L1, L2, L3, and L4 are intrafascicular electrodes. L3 and L4 may be activated for intraneural selectivity. Different fascicles may be activated without activating un-targeted fascicles. L1 and L2 may be activated for intrafascicular selectivity. Different subpopulations of nerve fibers may be activated within the same fascicle.

FIG. 2 shows a nerve representation of neurostimulation using extraneural electrodes.

FIG. 3 shows a block diagram of a neurostimulation system for interfascicular and intrafascicular selectivity.

FIGS. 4A-4B show block diagrams of two possible configurations for the stimulator, where the stimulator is an integrated/combined system (FIG. 4A) or two standalone systems that are wireless connected (FIG. 4B).

FIGS. 5A-5B show graphical user interfaces (GUIs) used for proof-of-concept studies.

FIG. 5A shows basic stimulation parameters input interface. FIG. 5B shows an App to apply experimental routine, including the duration of stimulation and rest period.

FIG. 6 illustrates a method of providing fascicular nerve fiber stimulation in accordance with various embodiments of the present disclosure set forth in Example 1.

FIG. 7 shows the results of the fascicular nerve fiber stimulation set forth in FIG. 6. Force data targeting two different electrodes in two different fascicles result in specific movement of the paw.

FIG. 8 illustrates another method of providing fascicular nerve fiber stimulation in accordance with various embodiments of the present disclosure set forth in Example 1.

FIGS. 9A-9B show the results of the fascicular nerve fiber stimulation set forth in FIG. 8. Shown are heat map representations of peak-to-peak amplitude of M-waves from HD-eEMG at the same physiological level.

FIGS. 10A-10B show data demonstrating selective intrafascicular nerve fiber stimulation by longitudinal intrafascicular electrodes (LIFEs).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Bioelectronic medicine approaches are used as an alternative to drug therapies to treat diseases. In these approaches, electrical stimulation is applied to the nerves to exert control over biological processes. The network of peripheral nerves presents potential for modulating and monitoring functions of internal organs and treating diseases. The nervous system functions by generating patterns of neural activity that underlie sensation and perception as well as control of movement, cardiovascular system, endocrine activity, immune response, and other physiological systems.

Anatomically, peripheral nerves are composed of one or more fascicles wrapped in epineural tissue. Each fascicle is included of groups of individual myelinated and/or unmyelinated nerve fibers wrapped in perineural tissue. The fibers inside of the fascicles can be majority motor or sensory, mixed, or autonomic fibers. Autonomic fibers include those carrying information to and from internal organs.

Because of the vast network of organs and functions that are carried out through the peripheral nerves, peripheral nerve stimulation (PNS) can be used as a treatment of various diseases (e.g., chronic pain, depression, and/or epilepsy), to restore motor functions, and to provide sensory feedback to people with prosthetics.

The peripheral nerve system consists of different nerves, such as sciatic nerves, vagus nerves and others. Specifically, the vagus nerve is the most extensively distributed cranial nerve in the body. It carries sensory and parasympathetic motor fibers connecting the heart, lungs, gastrointestinal tract, and brain. Because of its extensive distribution, researchers have been working on mapping the entire human vagus nerve using computational models and histology techniques.

Currently, vagus nerve stimulation (VNS) is FDA-approved for the treatment of epilepsy, depression and most recently, chronic ischemic stroke rehabilitation. Despite the efficacy of this bioelectronic medicine, the main challenge is the off-target activation induced by the lack of selectivity of the neurostimulation. The most common side effect of this treatment is activation of the neck muscle, followed by hoarseness. For this reason, researchers are seeking to increase the selectivity of VNS.

Most recently, computation models of VNS have been developed to create a quantitative framework that optimizes electrode location specific activation of nerve fibers governing intended effects versus unwanted side effects. Another study evaluated the morphology and tracked the type of fibers along the vagus nerve. The study also showed that fascicular vagus nerve stimulation asymmetrically elicits function- and organ-specific nerve potentials and physiological responses.

The efficacy of PNS in general, and specifically VNS, will depend on the ability to selectively stimulate the desired fibers and/or fascicles. Higher selectivity and specificity would achieve higher functionality without off-target activation. This selectivity can be divided into intraneural or fascicular selectivity and intrafascicular or subfascicular selectivity.

Intraneural selectivity describes the activation of a single fascicle in a nerve with more than one fascicle without activating the neighboring fascicles. On the other hand, intrafascicular selectivity consists of the activation of a subpopulation of nerve fibers within the same fascicle.

One of the key factors that affects selectivity is the bioelectronic interface utilized for the stimulation, in this case the type of electrode. Electrodes are classified as intraneural or extraneural electrodes. Intraneural electrodes include longitudinal intrafascicular electrodes (LIFEs), thin-film longitudinal intrafascicular electrodes (tf-LIFEs), transverse intrafascicular multichannel electrodes (TIMEs), or Utah slanted electrode array (UESEA). Unlike extraneural electrodes, intraneural electrodes provide direct access to neural fibers within a fascicle, which results in higher stimulation selectivity. In the case of LIFEs, they have been used to achieve higher selectivity. LIFEs are biocompatible and their minimalistic geometry allows the use of small current stimulus to selectively activate different types of nerves.

In sum, a need exists for more selective systems and methods for stimulating nerves. Numerous embodiments of the present disclosure aim to address the aforementioned need.

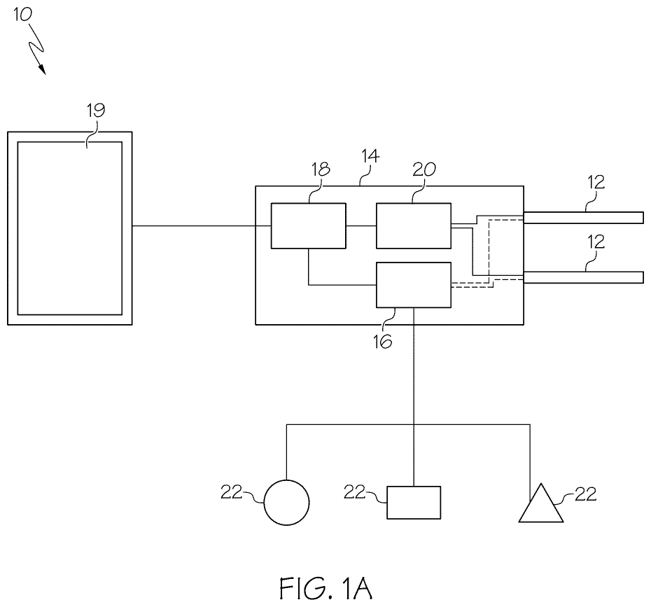

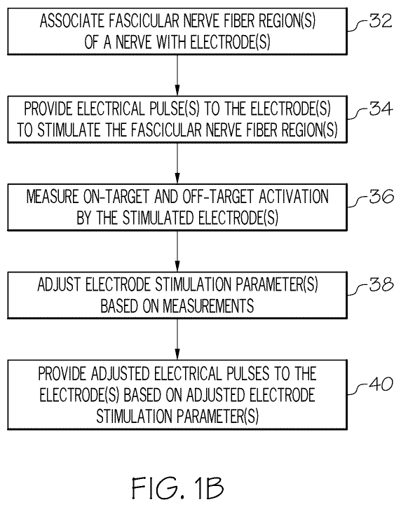

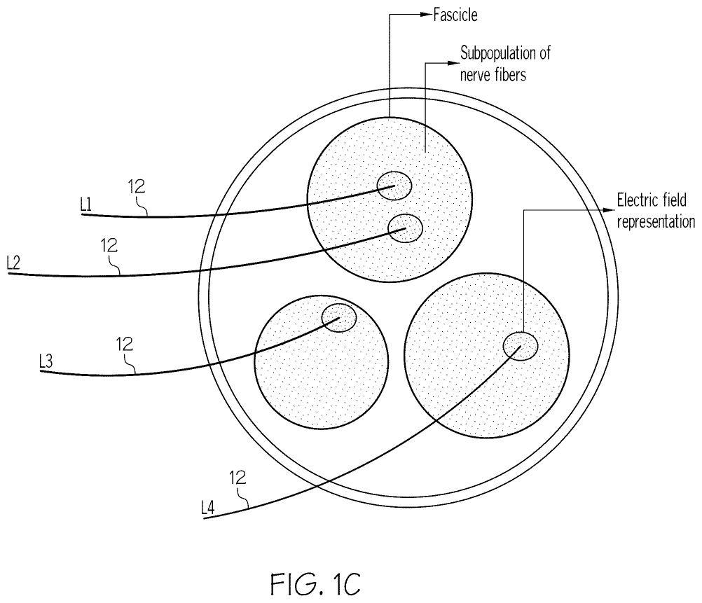

In some embodiments, the present disclosure pertains to a neurostimulation system operable for providing at least one fascicular nerve fiber stimulation to one or more nerves of a subject. The neurostimulation systems of the present disclosure may be illustrated as neurostimulation system 10 in FIG. 1A. In some embodiments, neurostimulation system 10 includes one or more electrodes 12 operational to associate with at least one fascicular nerve fiber region of a subject; a stimulator 14 in electrical communication with electrodes 12 and operational to provide one or more electrical pulses to electrodes 12 to stimulate at least one fascicular nerve fiber region; and a signal processing unit 16 in electrical communication with electrodes 12 and stimulator 14, and operational to measure on-target and off-target activation by the stimulated electrodes.

In some embodiments, stimulator 14 also includes a pulse processing unit 18 in electrical communication with signal processing unit 16. In some embodiments, pulse processing unit 18 is operational to adjust one or more electrode stimulation parameters based on measurements from signal processing unit 16.

In some embodiments, stimulator 14 further includes one or more channels connected to electrodes 12. In some embodiment, stimulator 14 also includes pulse generator 20 in electrical communication with pulse processing unit 18 and electrodes 12. In some embodiments, pulse generator 20 is operational to provide one or more adjusted electrical pulses to electrodes 12 based on one or more adjusted electrode stimulation parameters from pulse processing unit 18.

In some embodiments, pulse processing unit 18 further includes controller 19. In some embodiments, controller 19 is operational to control one or more stimulation parameters by electrodes 12.

Additional embodiments of the present disclosure pertain to methods of providing at least one fascicular nerve fiber stimulation to one or more nerves of a subject. In some embodiments, the methods of the present disclosure may utilize the neurostimulation systems of the present disclosure to provide the nerve fiber stimulation. With reference to FIGS. 1A and 1B for illustrative purposes, the methods of the present disclosure may include: (a) associating at least one fascicular nerve fiber region of a nerve with one or more electrodes 12 (Step 32 in FIG. 1B); (b) utilizing a stimulator 14 in electrical communication with the electrodes 12 to provide one or more electrical pulses to the electrodes 12 and thereby stimulate the fascicular nerve fiber region (Step 34 in FIG. 1B); and (c) utilizing a signal processing unit 16 in electrical communication with electrodes 12 and stimulator 14 to measure on-target and off-target activation by the stimulated electrodes 12 (Step 36 in FIG. 1B). In some embodiments, the methods of the present disclosure also include a step of utilizing a pulse processing unit 18 in electrical communication with signal processing unit 16 to adjust one or more electrode stimulation parameters based on measurements from signal processing unit 16 (Step 38 in FIG. 1B). In some embodiments, the methods of the present disclosure also include a step of utilizing a pulse generator 20 in electrical communication with pulse processing unit 18 to provide the adjusted electrical pulses to electrodes 12 based on one or more adjusted electrode stimulation parameters from pulse processing unit 18 (Step 40 in FIG. 1B).

As set forth in more detail herein, the methods and systems of the present disclosure can have numerous embodiments.

### Signal Processing Units

Signal processing units generally refer to processing units that are operational to measure on-target and off-target activation by stimulated electrodes. The methods and systems of the present disclosure may utilize various signal processing units.

For instance, in some embodiments, the signal processing units of the present disclosure are programmable. In some embodiments, the signal processing units of the present disclosure are in the form of a closed loop system. In some embodiments, the signal processing units of the present disclosure run on a computer, are embedded in a dedicated microcontroller, or are embedded on programmable logic, such as in a handheld device.

In some embodiments, signal processing unit 16 further includes one or more sensors 22 operational to assess physiological and neural responses from a subject due to neurostimulation. In some embodiments, signal processing unit 16 is operational to measure bio-signals from one or more sensors 22. In some embodiments, the sensors include, without limitation, an electrocardiogram (ECG) monitor, a heart rate monitor, a blood pressure monitor, a muscle activation monitor, a respirometer, a biochemical monitor, or combinations thereof.

In some embodiments, the methods of the present disclosure also include a step of measuring bio-signals from one or more sensors 22 in electrical communication with signal processing unit 16. In some embodiments, the methods of the present disclosure also include a step of utilizing the signals for adaptive or closed-loop control of on-target or off-target activation of stimulated electrodes.

### Pulse Processing Units

Pulse processing units generally refer to processing units that are operational to adjust one or more electrode stimulation parameters based on measurements from a signal processing unit. The methods and systems of the present disclosure may utilize various pulse processing units.

For instance, in some embodiments, the pulse processing units are programmable. In some embodiments, the pulse processing units are in the form of a closed loop system. In some embodiments, the pulse processing units can run on a computer, be embedded in a dedicated microcontroller, or be embedded on a programmable logic, such as in a handheld device.

In some embodiments, the pulse processing units of the present disclosure also include a controller 19 operational to control one or more stimulation parameters by electrodes 12. In some embodiments, the methods of the present disclosure also include a step of utilizing a controller associated with pulse processing unit 18 to control one or more stimulation parameters by electrodes 12.

In some embodiments, controller 19 includes a programming language. In some embodiments, the programming language includes programming instructions for controlling one or more stimulation parameters by electrodes 12.

In some embodiments, controller 19 includes a graphical user interface (GUI). In some embodiments, the GUI is operational to display one or more stimulation parameters by electrodes 12.

In some embodiments, controller 19 further includes one or more analog or digital controls operational for controlling one or more stimulation parameters by electrodes 12. In some embodiments, controller 19 is in the form of a programmed closed loop controller.

### Pulse Generators

Pulse generators generally refer to units operational to provide one or more adjusted electrical pulses to electrodes 12 based on one or more adjusted electrode stimulation parameters from pulse processing unit 18. The methods and systems of the present disclosure may utilize various pulse generators.

For instance, in some embodiments, pulse generator 20 produces a single or a train of pulses in different waveform shapes to stimulate fascicular nerve fibers. In some embodiments, pulse generator 20 can be composed of one or multiple channels connected to one or multiple electrodes.

In some embodiments, pulse generator 20 translates an analog or digital input to a desired neurostimulation parameter. In some embodiments, the stimulation parameters include, without limitation, waveform shape, stimulation amplitude, pulse width, frequency, single or train of pulses, stimulation duration, rest periods between stimulations, programmable experimental routines, single or multiple channels to send the stimulation, or combinations thereof.

### Electrodes

The methods and systems of the present disclosure may utilize various electrodes. For instance, in some embodiments, the electrodes include intrafascicular electrodes. In some embodiments, the electrodes include, without limitation, intrafascicular electrodes, longitudinal intrafascicular electrodes (LIFEs), thin-film longitudinal intra-fascicular electrodes (tf-LIFEs), transverse intrafascicular multichannel electrodes (TIMEs), multielectrode intrafascicular arrays (MEAs), Utah electrode arrays (UEA), Utah slanted electrode arrays (USEA), or combinations thereof.

Various methods may be utilized to associate fascicular nerve fiber regions of nerves with electrodes. For instance, in some embodiments, the association includes implanting fascicular nerve fiber regions of nerves with the electrodes. In some embodiments, the association includes associating a single electrode with a single fascicle of the nerve. In some embodiments, the electrode activates the single fascicle in the nerve with more than one fascicle without activating neighboring fascicles (e.g., L3 or L4 stimulation in FIG. 1C).

In some embodiments, the association includes associating a single electrode with each fascicle of a nerve. In some embodiments, the association includes associating a plurality of electrodes with a single fascicle of a nerve. In some embodiments, the plurality of electrodes activate a subpopulation of nerve fibers within the same fascicle (e.g., L1 and L2 stimulation in FIG. 1C). In some embodiments, the association includes associating at least two electrodes with a single fascicle of a nerve.

The methods and systems of the present disclosure may be utilized to stimulate various nerves. For instance, in some embodiments, the nerves to be stimulated include peripheral nerves. In some embodiments, the nerves include sciatic nerves. In some embodiments, the nerves include vagus nerves.

### Applications

The methods and systems of the present disclosure may have various applications. For instance, in some embodiments, the methods and systems of the present disclosure may be used to treat or prevent one or more conditions in the subject. In some embodiments, the one or more conditions include, without limitation, pain, chronic pain, neuropathic pain, phantom pain, depression, epilepsy, stroke, Parkinson's disease, Crohn's disease, obesity, cardiac vascular conditions, diabetes, bladder dysfunction, sexual disorders, or combinations thereof.

### Subjects

The methods and systems of the present disclosure may be utilized to stimulate nerves in various subjects. For instance, in some embodiments, the subject is a human being. In some embodiments, the subject is a non-human mammal. In some embodiments, the non-human mammal includes, without limitation, a horse, a rabbit, a mouse, a rat, a pig, a sheep, a cow, a dog, or a cat. In some embodiments, the non-human mammal is a domestic animal, such as a dog or a cat.

### Advantages

The methods and systems of the present disclosure can provide numerous advantages. For instance, in some embodiments, the methods and systems of the present disclosure improve the benefits of bioelectronic medicine using neurostimulation. In some embodiments, the methods and systems of the present disclosure reduce or remove side effects of neurostimulation by eliminating or minimizing off-target activation. As such, in some embodiments, the methods and systems of the present disclosure increase adherence to treatments because of the reduced or total removal of side effects.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Selective Stimulation of Peripheral Nerves with No or Reduced Off-Target Activation Using Intrafascicular Electrodes

This Example presents a system and methodology to selectively stimulate peripheral nerves with no or reduced off-target activation using intrafascicular electrodes. These offer higher selectivity, which will enable fascicular and subfascicular selectivity by activating neural fibers in a targeted fascicle or a subpopulation of nerve fibers within the same fascicle using intrafascicular electrodes with no or reduced off-target activation and improved therapeutical outcomes. This Example also provides a neurostimulation system that can achieve fascicular and subfascicular selectivity by activating neural fibers in a targeted fascicle or a subpopulation of nerve fibers within the fascicle using intrafascicular electrodes with no or reduced off-target activation.

The system and methods presented in this Example utilize a stimulator, electrodes for interfacing with the nerve, and the outcome measurements for on-target and off-target activation (somatosensory and biosignal response such as ECG, muscle activation, respiratory rate, and others). The stimulator can be a pulse processing unit and a pulse generator combined (e.g., a pacemaker) or standalone systems (e.g., a cochlear system). The stimulator will send the stimulation pulse using a front end-electrode interface. Single or multiple intrafascicular electrodes for neurostimulation can be implanted safely and selectively in the targeted fascicle.

The neurostimulation delivered by the system can achieve intraneural selectivity (e.g., fascicular selectivity, such as by implanting one intrafascicular electrode per fascicle, as shown in L3 and L4 in FIG. 1C). Additionally, the system allows selective activation of neural fibers inside the fascicle. For instance, the number of fibers activated can be graded (or titrated) by adjusting stimulation parameters.

Additionally, the system generates no or reduced off-target activation responsible for side effects, compared to other stimulation methods. Furthermore, the system can be utilized in a manner that does not generate undesired neural activity in fibers of neighboring fascicles.

The neurostimulation delivered by the system can also achieve intrafascicular selectivity (e.g., subfascicular selectivity, such as by implanting more than one intrafascicular electrode per fascicle, as shown in L1 and L2 in FIG. 1C). Additionally, the system allows one to activate selectively and gradually one or multiple subpopulations of neural fibers inside of the fascicle. The number of fibers activated can be graded (or titrated) by adjusting stimulation parameters.

Furthermore, the system generates no or reduced off-target activation responsible for side effects, compared to other stimulation methods. The physiological or neural responses are monitored to determine the on-target and off-target activation to adjust stimulation parameters.

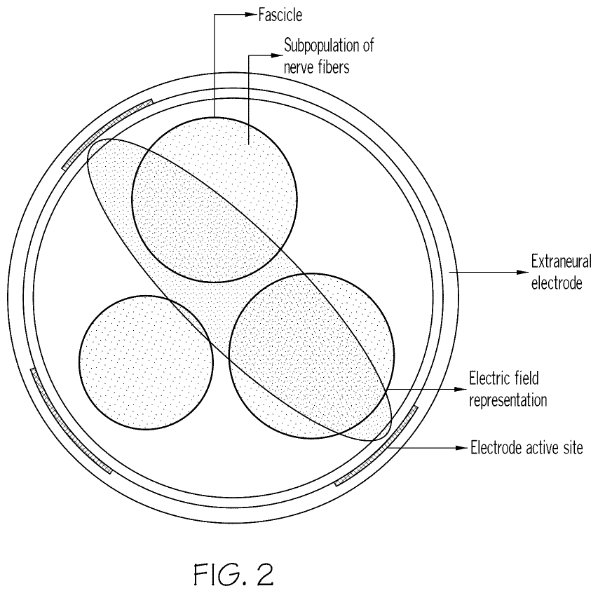

Additionally, this Example enables higher selectivity for neurostimulation than conventional extra-neural electrodes and systems (FIG. 2). This will translate into neurostimulation with no or reduced off-target activation limiting side effects of the bioelectronic technologies. The system provides precise timing and spatial selectivity that will enable the investigation of novel and adaptive bioelectronic therapies to treat complex impairments.

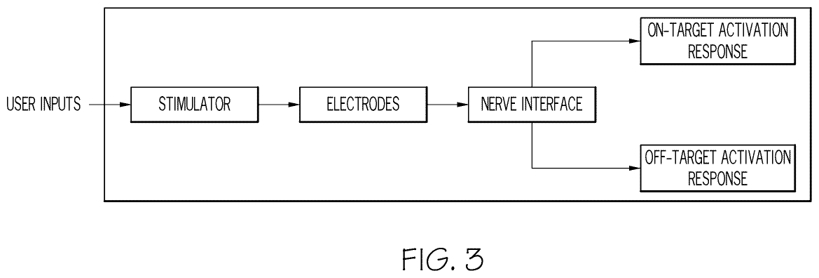

The presented system in this Example includes a neurostimulation system that can achieve fascicular and subfascicular selectivity by activating neural fibers in a targeted fascicle or a subpopulation of nerve fibers within the same fascicle using intrafascicular electrodes with no or reduced off-target activation (FIG. 3). The systems and methods presented in this Example include a stimulator, electrodes for interfacing with the nerve, and the outcome measurements for on-target and off-target activation (somatosensory and biosignal responses, such as electrocardiogram (ECG), heart rate, blood pressure, muscle activation, respiratory rate, and others).

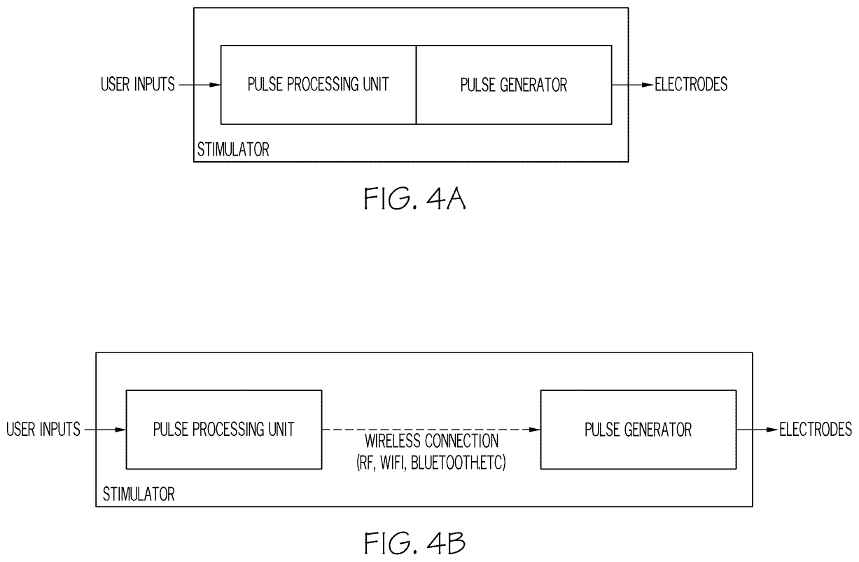

The stimulator includes a pulse processing unit and a pulse generator. These can be combined or integrated (e.g., a pacemaker) or standalone systems (e.g., a cochlear system) (FIGS. 4A-4B). The pulse processing unit can be a programmable, responsive and/or closed loop system. The pulse processing unit can transmit the signal to the pulse generator using wireless technology such as RF, Bluetooth, WiFi, or a wired connection. The pulse processing unit can have a controller that controls the parameters of the neurostimulation. The stimulation parameters can include waveform shape, stimulation amplitude, pulse width, frequency, single or train of pulses, stimulation duration, rest periods between trials, programmable experimental routines, or single or multiple channels to send the neurostimulation.

The controller can consist of a user interface that can be: (1) a programming/scripting language; (2) a graphical user interface (GUI); and/or (3) analog controls such as tactile switches and knobs.

The pulse processing unit can also read bio-signals from sensors that assess the physiological and neural responses from the subject due to the neurostimulation. These signals can be used for adaptive or closed-loop control of the targeted and/or off-target effects. The pulse processing unit can also include a programmed closed loop controller.

The pulse processing unit can run on a computer or can be embedded in a dedicated microcontroller or on programmable logic in a handheld device. The pulse generator produces a single or a train of pulses in different waveform shapes to activate neural fibers in a targeted fascicle or a subpopulation of nerve fibers within the same fascicle. The pulse generator translates an analog or digital input to the desired neurostimulation parameters entered by the user and/or the closed loop controller information. The pulse generator can be composed of one or multiple channels connected to one or multiple intrafascicular electrodes.

The stimulation waveforms are biphasic charge-balanced waveforms such as a sinewave, a square wave, a rectangular wave, or others. In this configuration, inputs to the pulse generator from the pulse processing unit consist in stimulation parameters for each channel and intrafascicular electrode.

The pulse generator can be any device that can be configured to accurately generate the specified stimulation parameters. The generator can be based on a commercially available device such as a programmable implantable neurostimulator or a benchtop/external/standalone neuro-stimulating system.

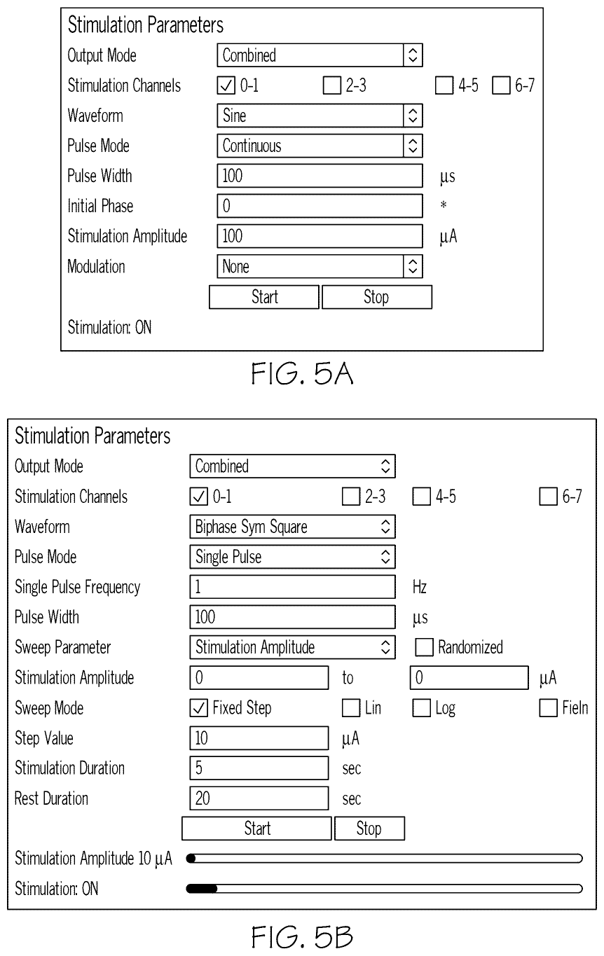

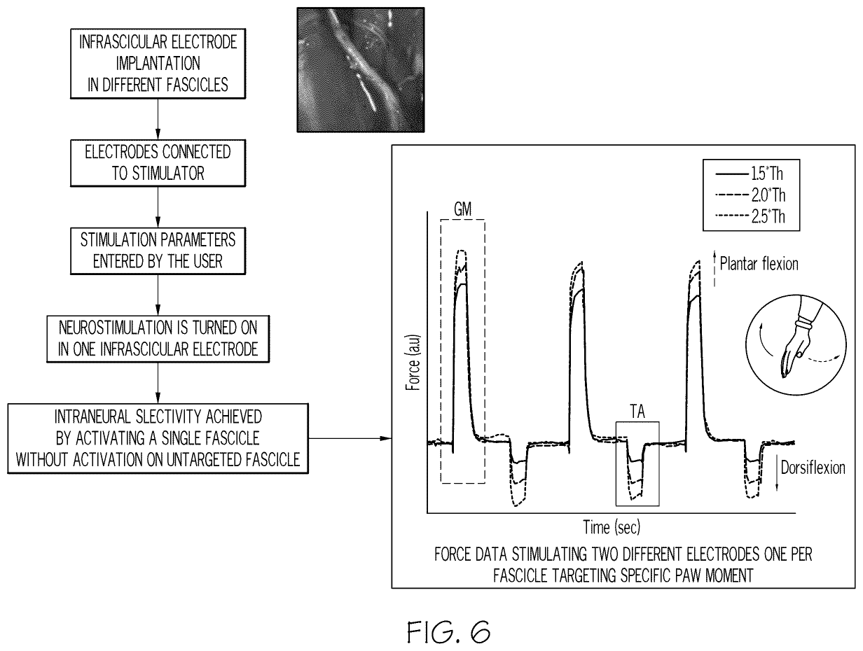

For proof-of-concept studies, the pulse processing unit interface was based on a python API developed for the control of the neurostimulator. The user-interface was based on a custom python-based GUI connected to the API via a python routine scheduler (FIGS. 5A-5B). The pulse generator used was a self-contained computer-controlled neurostimulator system.

The intrafascicular electrode can be any type of intraneural electrode that can selectively target one or multiple fascicles. The electrode includes longitudinal intrafascicular electrodes (LIFEs), thin-film longitudinal intra-fascicular electrodes (tf-LIFEs), transverse intrafascicular multichannel electrodes (TIMEs), and multielectrode arrays (MEAs) such as Utah electrode array (UEA) and Utah slanted electrode array (USEA).

For proof-of-concept studies, LIFEs were utilized to demonstrate the feasibility of intraneural and intrafascicular selectivity in neurostimulation of peripheral nerves. The minimalistic geometry of LIFEs allows the use of small current stimulus to activate nerve fibers. LIFEs are made from an insulated wire with a diameter thinner than a human hair. Due to their physical characteristics, LIFEs can be implanted inside the fascicles parallel to the nerve fibers and multiple LIFEs can be implanted in the same fascicle. The small active electrode site allows delivery of low levels electrical stimulation from the pulse generator to a subpopulation of nerve fibers inside the fascicles. LIFEs can be fabricated of materials that are biocompatible and highly flexible.

Another aspect of this Example includes methods to achieve intraneural and intrafascicular selectivity (fascicular and subfascicular selectivity). These methods include surgery procedure and data collection from somatosensory and physiological responses.

One or multiple intrafascicular electrodes are implanted inside peripheral nerves targeting specific fascicles. One or multiple intrafascicular electrodes are connected to the stimulator. The intrafascicular electrodes are electrically coupled to one or multiple pulse generators. The stimulation can also be applied to one or multiple intrafascicular electrodes. The stimulation pulse delivered will be the amount of charge required to activate neural fibers in a targeted fascicle or a subpopulation of nerve fibers within the same fascicle while producing little or no off-target activation. The neurostimulation can be delivered in a manner that does not generate undesired activity in fibers that are located in untargeted fascicles. Neurostimulation can occur by different methods.

### Example 1.1. Neurostimulation to Achieve Intraneural Selectivity

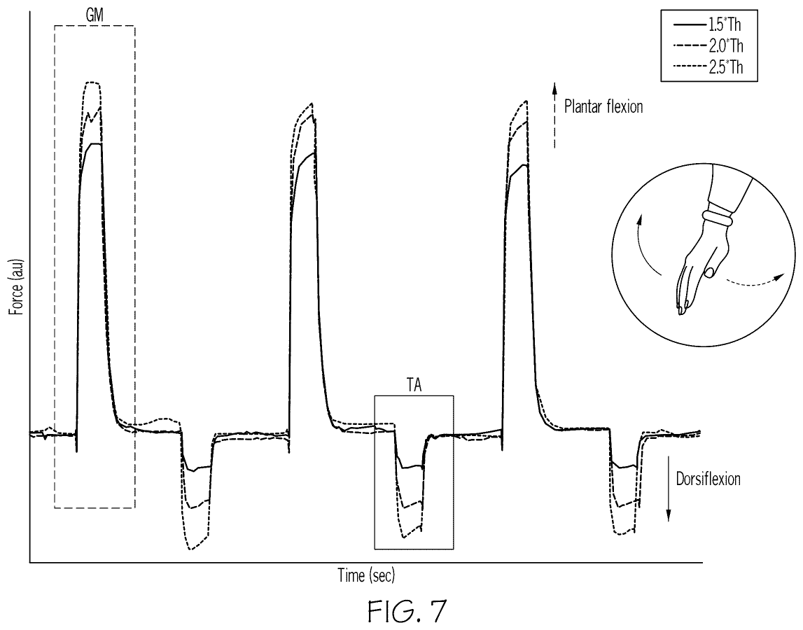

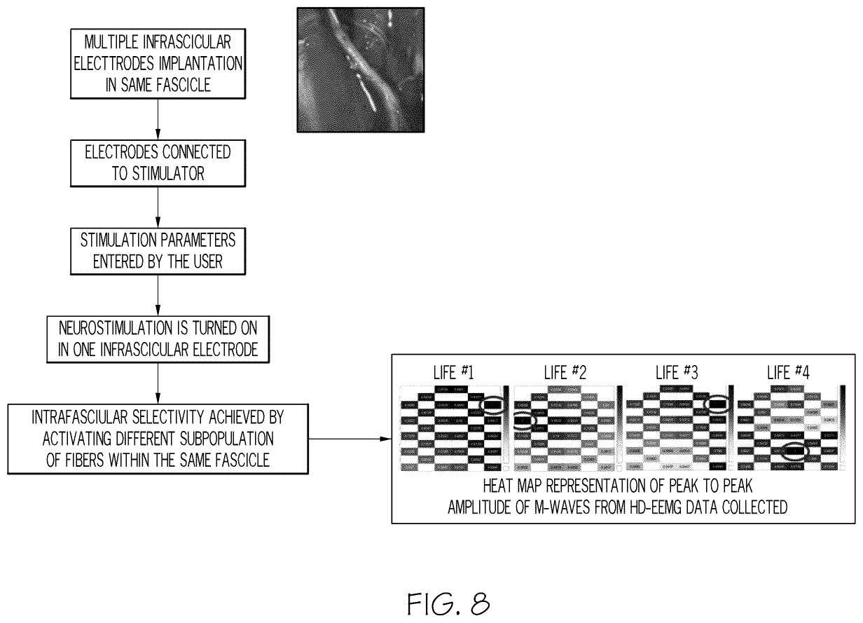

As illustrated in FIG. 6, this Example describes neurostimulation to achieve intraneural selectivity (i.e., fascicular selectivity). One intrafascicular electrode is implanted in a single fascicle of the nerve. If the nerve is multi-fascicular, one intrafascicular electrode can be implanted per fascicle. Biphasic balance charged pulses are applied to achieve the electrodes. Each electrode can activate neural fibers in the targeted fascicle with no or reduced off-target activation. The fascicles that are not required or desired to be activated can be avoided by appropriate selection of stimulation parameters (FIG. 7).

### Example 1.2. Neurostimulation to Achieve Intrafascicular Selectivity

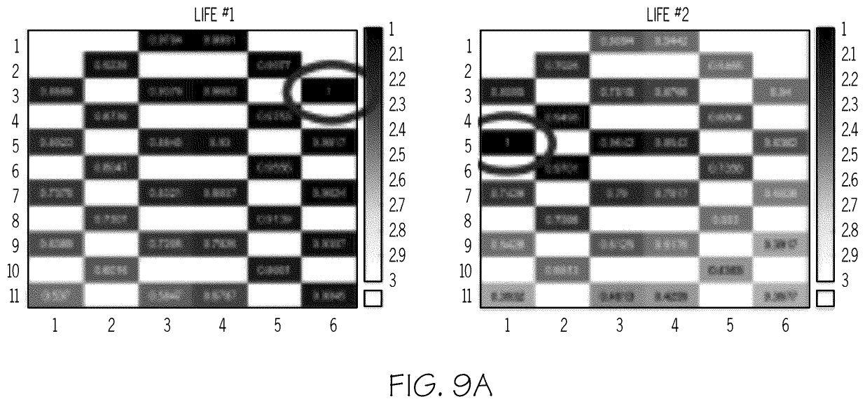

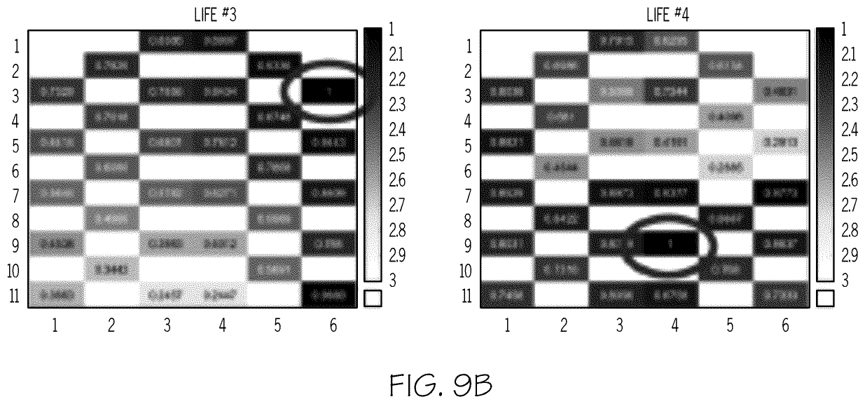

As illustrated in FIG. 8, this Example describes neurostimulation to achieve intrafascicular selectivity (i.e., subfascicular selectivity). Multiple intrafascicular electrodes are implanted in the same fascicle. Biphasic balance charged pulses are applied through the electrodes. Each electrode can activate a selective subpopulation of nerve fibers within the fascicle with no or reduced off-target activation. The subpopulation that is not required or desired to be activated can be avoided by selecting the specific electrode and the stimulation parameters. For example, four (4) intrafascicular electrodes are implanted in the same fascicle in the sciatic nerve and when the neurostimulation is applied through each one of the electrodes, the fibers that are activated are different (FIGS. 9A-9B).

Off target and on target activation is monitored using somatosensory and physiological response signals. These can include heart rate (HR), electrocardiogram (ECG), blood pressure (BP), respiratory rate (RR), muscle activity though high density or simple electromyography (HD-EMG or EMG), electroneugrams (ENG) and/or force recordings. These biomarkers can be collected with invasive or noninvasive procedures.

### Example 1.3. Experimental Results

Rodents (400-500 gr) were anesthetized using isoflurane gas (1.5-2.5% in medical-grade Oxygen). The level of anesthesia was periodically assessed with toe pinch, observation of eye blink. Body temperature was monitored via rectal thermometer and regulated with heat pads placed under the body of the animal. Heart rate, respiration rate, and SpO₂ were monitored continually using a vital signs monitor (Smith Medical, Model: Surgivet® Advisor@Vital Sign Monitor, OH, USA). The well-being of rats was maintained by administering saline subcutaneously to prevent dehydration every hour.

With the animal placed in supine position, the sternal notch is palpated. A V incision is then made and a blunt dissection is performed to separate the fascia and visualize the mandibular salivary glands. The glands are either dissected or retracted to visualize deeper structures. The muscles/skin were retracted using elastic stays to expose and have access to the nerve. The left vagus nerve was isolated from the left carotid artery. The nerve can be stabilized in a manner suitable to the surgeon. For off-target activation assessment, an extraneural electrode (hook or cuff electrode) was used to stimulate, which was wrapped around the nerve. For on-target activation assessment, after the nerve was stabilized, one or two LIFE electrodes were implanted.

The sciatic nerve was exposed and prepared by scalping and removing the fascia of the left hindlimb of the animal, and then a lateral incision of the thigh was performed. The muscles of the biceps femoris and gluteus superficialis were separated through minimal dissection techniques. The muscles were retracted using elastic stays to expose and have access to the nerve. The nerve was isolated from the surrounding connective tissue using tweezers N3, and an epineural dissection was used to expose the fascicles and visualize the bands of Fontana. The nerve was stabilized in a manner suitable for the surgeon.

LIFEs were implanted in both the tibial and the common peroneal fascicle of the sciatic nerve. For each rat, 2 to 4 intrafascicular electrodes were implanted per fascicle.

For both procedures, after implantation, neurostimulation was applied to induce neural activity in each fascicle or subpopulation of neural fibers within the same fascicle. The electrodes were secured to the epineurium proximally and distally with 8-0 non-absorbable sutures.

High Density epimysial Electromyography (HD-eEMG) was used to assess the selectivity of the neurostimulation. After the neurostimulation, the muscle response, the evoked potential, or M-waves, was recorded from each channel. Data was processed by filtering and doing a spike triggered average (STA) analysis of the responses. Peak-to-peak values were calculated from these STA for each channel. Peak-to-peak value is the change from the highest point of the spike to the lowest point of the same spike. After collecting these values, the data from HD-eEMG was represented using these peak-to-peak values in a heatmap form to represent the muscle activity. All the data comparison across electrodes was done at the same physiological level of stimulation (e.g., 1xThreshold, etc.).

A custom neurostimulator was used for the proof of concept of intraneural and intrafascicular selectivity using intrafascicular electrodes in the peripheral nerves. The neurostimulator was connected to the LIFEs to deliver the stimulation pulses. To avoid DC contamination, a DC-blocking circuit adapter was added between the LIFEs and the neurostimulator.

Biomarkers for assessment included heart rate (HR), electrocardiogram (ECG), blood pressure (BP), respiratory rate (RR), muscle activity though high density or simple electromyography, somatosensory evoked potential recordings, and electroneugrams (ENG) force recordings or monitoring functions of end-organs innervated by the vagus nerve. These biomarkers can be collected with invasive or non-invasive procedures. For on-target activation, HR, BP, RR, ECG, ENG, and EMG were used to assess the neurostimulation. Each biomarker was compared to their baseline with no stimulation. On-target activation will be achieved if there is no or reduced activation of the intrinsic laryngeal muscles and there is change in the biomarkers higher than the normal unstimulated baseline variability over 5 cycles.

For off-target activation, a flexible electrode array was used to record HD-eEMG. This array was placed on top of the intrinsic laryngeal muscles of the rat. A needle electrode can also be used to record EMG. During the experiment, saline was added on top of the array to guarantee the hydration of the muscle. The electrode array was coupled to a Nano2 head stage (Ripple Neuro, Inc) and broadband data were sampled at 30 KS/s.

### Example 1.4. Sciatic Nerve Approach Assessment

For on-target activation assessment, intrafascicular selectivity can be determined using a 32-channel flexible electrode array (NeuroNexus Inc.), which can be used to record HD-eEMG from the muscle activity on the gastrocnemius lateralis of the rodent. For the placement of this array, and the muscle exposed, the electrode was placed from distal to proximal in the middle of the distance between the ankle and the knee. During the experiment, saline was added on top of the array to guarantee the hydration of the muscle. The electrode array was coupled to a Nano2 head stage (Ripple Neuro, Inc) and broadband data were sampled at 30 KS/s.

For on-target and off-target activation assessment, isometric force recording was also collected using a 6-axis force transducer (JR3, Inc., Woodland, CA, USA). To ensure isometric conditions and reliable data collection, the paw of the rodent was secured to the transducer using a custom-made 3D-printed boot. The baseline of the axis was adjusted using on-board trimmers. The data acquisition system (Scout processor, Ripple Neuro, USA) samples the output data at 30 kS/s. Data was processed and filtered using MATLAB or python.

### Example 1.4.1. Intraneural Selectivity—Fascicular Selectivity

A biphasic square pulse was applied to the intrafascicular electrode to elicit a physical response. The pulse amplitude of the stimulation was increased in a fixed step 2 uA or 4 uA. For each step, the neurostimulation was applied for 5 seconds at a frequency of 10 Hz. Then, a rest period of 20 seconds was applied to prevent muscle fatigue and to restore the “baseline” physiological response before the stimulation.

For off-target activation, one hook electrode was wrapped around the left vagus nerve. For rats, the vagus nerve has 1-2 fascicles. When the neurostimulation was applied to the nerve, ECG was recorded showing differences from the baseline (no stimulation). Visual inspection of the intrinsic laryngeal muscles showed activation when the neurostimulation was applied.

One intrafascicular electrode was implanted per fascicle in the sciatic nerve. One in the tibial and one in the peroneal fascicle. When the neurostimulation was applied to the peroneal fascicle, the force results showed a dorsiflexion movement (FIG. 7) with activation predominantly of the tibialis anterior muscle. For the neurostimulation in the tibial fascicle, the force results showed a plantar flexion movement. The M-waves responses of gastrocnemius lateralis were recorded using the HD-eEMG flexible electrode array.

For off-target activation assessment, when the intrafascicular electrode implanted in the tibial fascicle was used for the neurostimulation, there was no force data for dorsiflexion movement. When the amplitude was increased in steps, there was a gradual activation of the motor fibers that translated in force, which increased gradually and m-waves peak to peak amplitude gradually activated.

### Example 1.4.2. Intrafascicular Selectivity—Subfascicular Selectivity

A biphasic square pulse was applied to the intrafascicular electrode to elicit a physical response. The pulse amplitude of the stimulation was increased in a fixed step 2 uA or 4 uA. For each step, the neurostimulation was applied for 5 seconds at a frequency of 10 Hz. Then, a rest period of 20 seconds was applied to prevent muscle fatigue and/or to restore the “baseline” physiological response before the stimulation.

Multiple intrafascicular electrodes (2-4) were implanted in the tibial fascicle. Neurostimulation was applied to each electrode. HD-eEMG was recorded from the gastrocnemius lateralis. Data analysis of the M-waves showed that the regions of muscle activation are different across all intrafascicular electrodes. The data was compared at the same physiological levels (e.g., at threshold, at 1.5 threshold, etc.) (FIGS. 9A-9B).

The stimulation pulse amplitude was increased in fixed steps and the data shows that there was a gradual activation of the motor fibers that translated in force increased gradually and m-waves peak to peak amplitude were gradually activated. The neurostimulation of the intrafascicular electrode implanted in the tibial fascicle showed no force data for dorsiflexion movement.

### Example 1.5. Discussion

Bioelectronic medicine approaches are used as an alternative to drug therapies to treat diseases. In these approaches, electrical stimulation is applied to the nerves to exert control over biological processes that cause such diseases.

The network of peripheral nerves presents potential for modulating and monitoring functions of internal organs and treating diseases. The nervous system functions by generating patterns of neural activity that underlie sensation and perception as well as control of movement, cardiovascular system, endocrine activity, immune response, and other physiological systems.

Because of the vast network of organs and functions that are carried out through the peripheral nerves, peripheral nerve stimulation (PNS) is used as a treatment of chronic pain, depression, and epilepsy, to restore motor functions, provide sensory feedback to people with prosthetics, and others. The peripheral nerve system consists of different nerves, such as sciatic nerves, vagus nerves and others. Currently, vagus nerve stimulation is FDA-approved for the treatment of epilepsy, depression and most recently, chronic ischemic stroke rehabilitation. Despite the efficacy of this bioelectronic medicine, the main challenge is the side effects that could include activation of the neck muscles, hoarseness and others. For this reason, patients will stop using the treatments. This Example presents a system and methodology to selectively stimulate peripheral nerves removing or reducing side effects. Using this technology would improve the benefits of bioelectronic medicine using neurostimulation.

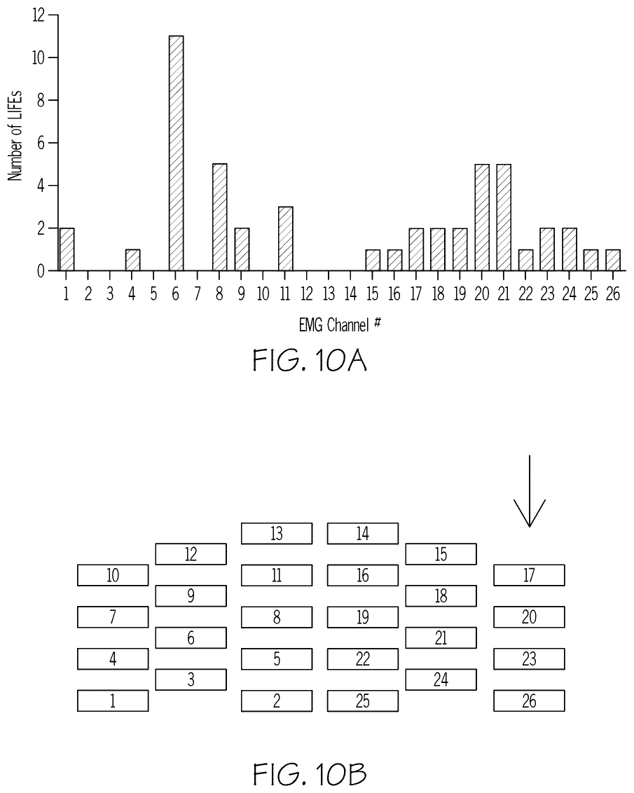

FIGS. 10A-10B show data demonstrating selective intrafascicular nerve fiber stimulation by longitudinal intrafascicular electrodes (LIFEs) across different channels. In particular, FIG. 10A shows the number of times a particular EMG channel was the spatial maximum (i.e., it was activated at a level that was higher than all other EMG channels). FIG. 10B shows the location of the EMG channels within the array. The arrow indicates the orientation of the muscle fibers with respect to the EMG channel array. Data are reported for all LIFEs (n=49) for stimulation at 1.5 times the threshold for that LIFE. For a given LIFE, the EMG channel that was the spatial maximum indicates preferential, or selective, stimulation of a particular region of the fascicle by that LIFE. The distribution of spatial maxima across the array in FIG. 10A demonstrates that different LIFEs preferentially and selectively activate different regions of the fascicle.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A neurostimulation system operable for providing at least one fascicular nerve fiber stimulation to one or more nerves of a subject, said system comprising:
(a) one or more electrodes operational to associate with at least one fascicular nerve fiber region of the subject;
(b) a stimulator in electrical communication with the one or more electrodes, wherein the stimulator is operational to provide one or more electrical pulses to the one or more electrodes and thereby stimulate at least one fascicular nerve fiber region; and
(c) a signal processing unit in electrical communication with the one or more electrodes and the stimulator, wherein the signal processing unit is operational to measure on-target and off-target activation by the stimulated electrodes.

2. The system of claim 1, wherein the stimulator further comprises:
a pulse processing unit in electrical communication with the signal processing unit, wherein the pulse processing unit is operational to adjust one or more electrode stimulation parameters based on measurements from the signal processing unit; and
a pulse generator in electrical communication with the pulse processing unit and one or more electrodes, wherein the pulse generator is operational to provide the one or more adjusted electrical pulses to the one or more electrodes based on one or more adjusted electrode stimulation parameters from the pulse processing unit.

3. The system of claim 2, wherein the pulse processing unit further comprises a controller, wherein the controller is operational to control one or more stimulation parameters by the one or more electrodes.

4. The system of claim 3, wherein the controller comprises a graphical user interface (GUI), wherein the GUI is operational to display one or more stimulation parameters by the one or more electrodes.

5. The system of claim 1, wherein the signal processing unit further comprises one or more sensors operational to assess physiological and neural responses from the subject due to the neurostimulation, and wherein the signal processing unit is operational to measure bio-signals from the one or more sensors.

6. The system of claim 5, wherein the one or more sensors are selected from the group consisting of an electrocardiogram (ECG) monitor, a heart rate monitor, a blood pressure monitor, a muscle activation monitor, a respirometer, a biochemical monitor, or combinations thereof.

7. The system of claim 1, wherein the pulse processing unit and the signal processing unit are in the form of a closed loop system.

8. The system of claim 1, wherein the one or more electrodes comprise intrafascicular electrodes.

9. The system of claim 1, wherein the one or more electrodes are selected from the group consisting of intrafascicular electrodes, longitudinal intrafascicular electrodes (LIFEs), thin-film longitudinal intra-fascicular electrodes (tf-LIFEs), transverse intrafascicular multichannel electrodes (TIMEs), multielectrode intrafascicular arrays (MEAs) Utah electrode arrays (UEA), Utah slanted electrode arrays (USEA), or combinations thereof.

10. A method of providing at least one fascicular nerve fiber stimulation to one or more nerves of a subject, said method comprising:
(a) associating the at least one fascicular nerve fiber region of the nerve with one or more electrodes;
(b) utilizing a stimulator in electrical communication with the one or more electrodes to provide one or more electrical pulses to the one or more electrodes and thereby stimulate the at least one fascicular nerve fiber region; and
(c) utilizing a signal processing unit in electrical communication with the one or more electrodes and the stimulator to measure on-target and off-target activation by the stimulated electrodes.

11. The method of claim 10, further comprising a step of utilizing a pulse processing unit in electrical communication with the signal processing unit to adjust one or more electrode stimulation parameters based on measurements from the signal processing unit.

12. The method of claim 11, further comprising a step of utilizing a pulse generator in electrical communication with the pulse processing unit and the one or more electrodes to provide the one or more adjusted electrical pulses to the one or more electrodes based on one or more adjusted electrode stimulation parameters from the pulse processing unit.

13. The method of claim 12, wherein the one or more stimulation parameters are selected from the group consisting of waveform shape, stimulation amplitude, pulse width, frequency, single or train of pulses, stimulation duration, rest periods between stimulations, programmable experimental routines, single or multiple channels to send the stimulation, or combinations thereof.

14. The method of claim 12, further comprising a step of utilizing a controller associated with the pulse processing unit to control one or more stimulation parameters by the one or more electrodes.

15. The method of claim 14, further comprising a step of measuring bio-signals from one or more sensors in electrical communication with the signal processing unit.

16. The method of claim 10, wherein the associating comprises associating a single electrode with a single fascicle of the nerve.

17. The method of claim 16, wherein the electrode activates the single fascicle in the nerve with more than one fascicle without activating neighboring fascicles.

18. The method of claim 10, wherein the associating comprises associating a plurality of electrodes with a single fascicle of the nerve.

19. The method of claim 18, wherein the plurality of electrodes activates a subpopulation of nerve fibers within the same fascicle.

20. The method of claim 10, wherein the one or more nerves comprise peripheral nerves.

21. The method of claim 10, wherein the one or more nerves comprise sciatic nerves.

22. The method of claim 10, wherein the one or more nerves comprise vagus nerves.

23. The method of claim 10, wherein the method is used to treat or prevent one or more conditions in the subject.

24. The method of claim 23, wherein the one or more conditions are selected from the group consisting of pain, chronic pain, neuropathic pain, phantom pain, depression, epilepsy, stroke, Parkinson's disease, Crohn's disease, obesity, cardiac vascular conditions, diabetes, bladder dysfunction, sexual disorders, or combinations thereof.

25. The method of claim 10, wherein the subject is a human being.
